(19) United States
(12) Patent Application Publication
(10) Pub. No.: US 2003/0059857 A1
(43) Pub. Date: Mar. 27, 2003

(54) G-protein coupled receptor and methods

(75) Inventors: Fang L. Zhang, Fanwood, NJ (US); Lin Luo, Edison, NJ (US)

Correspondence Address: SCHERING-PLOUGH CORPORATION, PATENT DEPARTMENT (K-6-1, 1990), 2000 GALLOPING HILL ROAD, KENILWORTH, NJ, 07033-0530, US

(73) Assignee: Schering Corporation

(21) Appl. No.: 10/243,106

(22) Filed: Sep. 13, 2002

Related U.S. Application Data
(60) Provisional application No. 60/322,668, filed on Sep. 17, 2001.

(51) Int. Cl. G01N 33/567; A61K 39/395; A61K 39/385; A61K 31/00

(52) U.S. Cl. 435/7.21; 424/143.1; 514/1

(57) ABSTRACT

The present invention provides an isolated mammalian SP174 receptor and ligands of the receptor. This invention further provides screening methods for identifying specific agonists and antagonists of the mammalian SP174 receptor. Also provided are agonists and antagonists of the SP174 receptor and methods useful for treating medical conditions caused or mediated by the SP174 receptor.

### TECHNICAL FIELD

The present invention relates to a G-protein coupled receptor and the identification of ligands that bind to the receptor. More particularly, it relates to methods of using the receptor in screening systems to identify agonists and antagonists of the receptor useful for the treatment of various medical conditions. This invention further relates to agonists and antagonists of the receptor.

### BACKGROUND OF THE INVENTION

GPCRs (G-protein coupled receptors) form a superfamily of integral plasma membrane proteins that respond to a wide range of ligands, including biogenic amines, lipids, protein hormones, peptides, proteases and divalent cations. These receptors also play an important role in sensory perception including vision and olfaction. GPCRs, therefore, represent a primary mechanism by which cells respond to alterations in their external environment. The members of the GPCR superfamily are related both structurally and functionally. The receptors contain seven distinct hydrophobic domains, which are highly conserved and are linked by hydrophilic amino acid sequences of varied length. Agonist binding stimulates GPCRs to transmit signals across the plasma membrane through an interaction with heterotrimeric G-proteins. This interaction leads to changes in intracellular levels of second messenger molecules such as calcium or cAMP, which subsequently modulate numerous signaling pathways culminating in the physiological response of the cell to the external stimuli.

Since the initial cloning of GPCR gene sequences over a decade ago, more than 300 G-protein coupled receptors have been identified. More than 100 of these are called “orphan” receptors because their endogenous ligands have not yet been identified. Recently, several novel neuropeptides have been identified through their ability to activate orphan GPCRs (oGPCRs). These include nociceptin, the orexins and prolactin-releasing peptide. The identification of these novel ligand/receptor pairs has opened up new areas of biology and new opportunities for drug discovery.

Since the endogenous ligand of SP174 was idenified to be nucleotide ADP, SP174 belongs to the class of P2Y purinergic receptors. P2Y receptors are a growing class of receptors whose endogenous ligands are purine or pyrimidine nucleoides. These receptors have broad distribution in mammalian tissues. The nucleotides have been shown to be transmitter in sympathetic nerves, parasympathetic nerves, sensory motor nerves, intramural nerves and synaptic transmission in ganglia and brain. SP174 is homologous to SP1999, which has been identified as the ADP receptor in blood platelets aggregation. SP1999 is the target of cardiovascular drug clopidogrel. Therefore, SP174 has similar function in platelet aggregation, and SP174 may be used as a drug target to develop cardiovascular drugs. Expression analysis of SP174 shows that it was mainly expressed in immune tissues and CNS tissues, which indicates that SP174 may be useful to develop immunological and neurological therapies.

Such an expression pattern points to the possible role of the SP174 receptor in neurodegenerative disorders such as Parkinson's, Alzheimer's, Huntington's, amyotrophic lateral sclerosis (ALS) and multiple sclerosis (MS). Accordingly, the identification of the ligands for the SP174 receptor will provide critical tools necessary to evaluate the precise role of the SP174 receptor in such medical conditions. In addition, identification of the ligands for the SP174 receptor is an important first step in the development of agonists and antagonists of the receptor. The availability of such reagents will be useful in determining the physiological role of the SP174 receptor and facilitate the development of novel therapeutic agents for the treatment of medical conditions mediated by the binding of the SP174 receptor to its ligands.

### SUMMARY OF THE INVENTION

The present invention fills the foregoing needs by identifying SP174 receptor ligands and, more particularly, providing methods for identifying agonists and antagonists of the SP174 receptor comprising:

(a) contacting a mammalian SP174 receptor or a functional fragment thereof in the presence of a known amount of a detectable SP174 receptor ligand with a sample to be tested for the presence of the SP174 receptor agonist or antagonist; and

(b) measuring the amount of the detectable SP174 ligand specifically bound to the receptor;

whereby the SP174 receptor agonist or antagonist in the sample is identified by measuring substantially reduced binding of the detectable ligand to the SP174 receptor, compared to what would be measured in the absence of such agonist or antagonist. Preferably, the SP174 receptor is mammalian and more preferably human. Most preferably, the SP174 receptor is characterized by having the amino acid sequence of SEQ ID NO: 2. Preferably, the SP174 receptor ligand is selected from the group consisting of ADP, ADPβS, 2-MeS-ADP, 2-MeS-ATP, 2-Cl-ATP, ATPγS, Ap3A, IDP and analogs thereof.

In a preferred embodiment, membranes isolated from cells expressing a nucleic acid encoding the SP174 receptor are used as the source of the receptor.

The present invention also relates to antagonists of the SP174 receptor that specifically bind to the SP174 receptor. The SP174 receptor antagonists of this invention include small organic molecules, peptides, inhibitory ligand analogs, and antibodies or antigen binding fragments of antibodies, which specifically bind to the SP174 receptor. In a preferred embodiment, the SP174 receptor antagonists are orally active, small organic molecules.

This invention also relates to agonists of the SP174 receptor. The SP174 receptor agonists of this invention include small organic molecules, peptides and ligand analogs that specifically bind to a SP174 receptor. In a preferred embodiment the SP174 receptor agonists are orally active, small organic molecules.

This invention also contemplates pharmaceutical compositions, for use in treating the SP174 receptor mediated medical conditions, comprising:

(a) an effective amount of an SP174 receptor agonist or antagonist; and

(b) a pharmaceutically acceptable carrier.

This invention further provides a method for treating a subject afflicted with a medical condition caused or mediated by the SP174 receptor comprising administering a pharmaceutical composition comprising: (a) an effective amount of an agonist or antagonist of the SP174 receptor; and (b) a pharmaceutically acceptable carrier. Preferably, the subject is a mammal; more preferably the mammal is a human being. In a preferred embodiment, the medical condition is a neurodegenerative disease such as Parkinson's, Alzheimer's, Huntington's, amyotrophic lateral sclerosis (ALS) or multiple sclerosis (MS). Alternatively, the medical condition may be related to cardiovascular disease or a condition related to platelet aggregation.

### DESCRIPTION OF THE INVENTION

All references cited herein are hereby incorporated herein in their entirety by reference.

SP174 Ligand Identification

The SP174 receptor sequence is disclosed in PCT Publication WO 96/30406 and referred to therein as human G-protein coupled receptor 3 or GPR3. The nucleotide sequence of the complete open reading frame (ORF) and the corresponding amino acid sequence of human SP174 receptor cDNA are defined in the Sequence Listing by SEQ ID NO: 1 and SEQ ID NO: 2, respectively. The SP174 receptor sequence is 1002 nucleotides long with an ORF encoding 333 amino acid residues. The hydrophilicity profile of the deduced peptide sequences revealed the presence of seven hydrophobic regions, consistent with a seven transmembrane structure typical of G-protein coupled receptors.

Dot blot analysis using a probe derived from the SP174 receptor sequence indicated that the receptor was expressed in the central nervous system and in the spleen, liver, peripheral leukocytes, bone marrow and placenta. Northern blot and in-situ hybridization experiments further pointed to expression in substantia nigra, spinal cord, medulla, hippocampus, putamen, amygdala, caudate, thalamus and subthalamic nodes.

In order to identify SP174 receptor ligands the SP174 open reading frame was subcloned into the pcDNA3.1 expression vector under the control of the CMV promoter and transfected into mammalian cells. After allowing two days for receptor expression, the transfected cells were seeded into 96 well plates, loaded with the Ca²⁺ sensitive Fluo-3AM dye and challenged with various compounds. The peak increments in Ca²⁺ evoked by the test compounds were measured using the Fluorometric Imaging Plate Reader (FLIPR) system. The endogenous ligand of the SP174 receptor was purified from rat spinal cords using a series of chromatography steps and its structure was identified as being ADP by Mass spectroscopy. ADP was shown to activate the SP174 receptor through inhibition of adenylyl cyclase. In addition to ADP, other agonists were also identified for SP174. The EC50 for ADP is 1.6 nM, ADPβS is 8 nM, 2-MeS-ADP is 0.32 nM, 2-MeS-ATP is 8 nM, 2-Cl-ATP is 8 nM, ATP is 0.32 nM, AP3A is 8 nM, IDP is 200 nM and ATPγS is 8 nM, and the order of potency was determined to be: 2-MeS-ADP=ATP>ADP>ADPβS=ATPγS=2-Cl-ATP=2-MeS-ATP=AP3A>IDP.

The pharmacology of SP174 was the characteristic of P2Y receptor class. P2Y receptors are a class of G-protein coupled receptors activated primarily by ATP, ADP, UTP and UDP. Five mammalian P2Y receptors have been cloned so far including P2Y1, P2Y2, P2Y4, P2Y6 and P2Y11. These receptors share high homology to each other. P2Y1, P2Y2, P2Y4, and P2Y6 couple to the activation of phospholipase C (PLC) while P2Y11 couples to the stimulation of both PLC and the adenylyl cyclase pathways. P2Y1, P2Y2, and P2Y11 are selectively activated by ATP. Whereas, P2Y6 is selectively activated by UDP and P2Y4 can be selectively activated by both ATP and UTP. ADP receptors have been identified from C6 rat glioma cells and human blood platelets but have not been cloned. The ADP receptor in C6 cells couples to the inhibition of adenylyl cyclase, and the two ADP receptors in platelets couple to inhibition of cAMP and PLC pathways respectively.

SP174 Receptor Analogs

The term “analog(s)” means a mammalian SP174 receptor of the invention which has been modified by deletion, addition, modification or substitution of one or more amino acid residues in the wild-type receptor. It encompasses allelic and polymorphic variants, and also muteins and fusion proteins which comprise all or a significant part of such a mammalian SP174 receptor, e.g., covalently linked via a side-chain group or terminal residue to a different protein, polypeptide or moiety (fusion partner).

Some amino acid substitutions are preferably “conservative”, with residues replaced with physicochemically similar residues, such as Gly/Ala, Asp/Glu, Val/Ile/Leu, Lys/Arg, Asn/Gln and Phe/Trp/Tyr. Analogs having such conservative substitutions typically retain substantial the SP174 receptor binding activity. Other analogs, which have non-conservative substitutions such as Asn/Glu, Val/Tyr and His/Glu, may substantially lack such activity. Nevertheless, such analogs are useful because they can be used as antigens to elicit production of antibodies in an immunologically competent host. Because these analogs retain many of the epitopes (antigenic determinants) of the wild-type receptors from which they are derived, many antibodies produced against them can also bind to the active-conformation or denatured wild-type receptors. Accordingly, such antibodies can also be used, e.g., for the immunopurification or immunoassay of the wild-type receptors.

Some analogs are truncated variants in which residues have been successively deleted from the amino- and/or carboxyl-termini, while substantially retaining the characteristic ligand binding activity.

Modifications of amino acid residues may include but are not limited to aliphatic esters or amides of the carboxyl terminus or of residues containing carboxyl side chains, O-acyl derivatives of hydroxyl group-containing residues, and N-acyl derivatives of the amino-terminal amino acid or amino-group containing residues, e.g., lysine or arginine.

Other analogs are mammalian SP174 receptors containing modifications, such as incorporation of unnatural amino acid residues, or phosphorylated amino acid residues such as phosphotyrosine, phosphoserine or phosphothreonine residues. Other potential modifications include sulfonation, biotinylation, or the addition of other moieties, particularly those which have molecular shapes similar to phosphate groups.

Analogs of the mammalian SP174 receptors can be prepared by chemical synthesis or by using site-directed mutagenesis [Gillman et al., Gene 8:81 (1979); Roberts et al., Nature 328:731 (1987) or Innis (Ed.), 1990, PCR Protocols: A Guide to Methods and Applications, Academic Press, New York, N.Y.] or by the polymerase chain reaction method [PCR; Saiki et al., Science 239:487 (1988)], as exemplified by Daugherty et al. [Nucleic Acids Res. 19:2471 (1991)] in order to modify nucleic acids encoding the complete receptors. Adding epitope tags for purification or detection of recombinant products is envisioned.

General techniques for nucleic acid manipulation and expression that can be used to make the analogs are described generally, e.g., in Sambrook, et al., Molecular Cloning: A Laboratory Manual (2d ed.), 1989, Vols. 1-3, Cold Spring Harbor Laboratory. Techniques for the synthesis of polypeptides are described, for example, in Merrifield, J. Amer. Chem. Soc. 85:2149 (1963); Merrifield, Science 232:341 (1986); and Atherton et al., Solid Phase Peptide Synthesis: A Practical Approach, 1989, IRL Press, Oxford.

Still other analogs are prepared by the use of agents known in the art for their usefulness in cross-linking proteins through reactive side groups. Preferred derivatization sites with cross-linking agents are free amino groups, carbohydrate moieties and cysteine residues.

Substantial retention of ligand binding activity by the foregoing analogs of the mammalian SP174 receptor typically entails retention of at least about 50%, preferably at least about 75%, more preferably at least about 80%, and most preferably at least about 90% of the SP174 receptor binding activity and/or specificity of the corresponding wild-type receptor.

Some of the physical variants have substantial amino acid sequence homology with the amino acid sequences of the mammalian SP174 receptors or polypeptides. In this invention, amino acid sequence homology, or sequence identity, is determined by optimizing residue matches and, if necessary, by introducing gaps as required. Homologous amino acid sequences are typically intended to include natural allelic, polymorphic and interspecies variations in each respective sequence.

Typical homologous proteins or peptides will have from 25-100% homology (if gaps can be introduced) to 50-100% homology (if conservative substitutions are included), with the amino acid sequence of the SP174 receptors. Primate species receptors are of particular interest.

Observed homologies will typically be at least about 35%, preferably at least about 50%, more preferably at least about 75%, and most preferably at least about 80% or more. See Needleham et al., J. Mol. Biol. 48:443-453 (1970); Sankoff et al. in Time Warps, String Edits, and Macromolecules: The Theory and Practice of Sequence Comparison, 1983, Addison-Wesley, Reading, Mass.; and software packages from IntelliGenetics, Mountain View, Calif., and the University of Wisconsin Genetics Computer Group, Madison, Wis.

Glycosylation variants include, e.g., analogs made by modifying glycosylation patterns during synthesis and processing in various alternative eukaryotic host expression systems, or during further processing steps. Particularly preferred methods for producing glycosylation modifications include exposing the mammalian the SP174 receptors to glycosylating enzymes derived from cells which normally carry out such processing, such as mammalian glycosylation enzymes. Alternatively, deglycosylation enzymes can be used to remove carbohydrates attached during production in eukaryotic expression systems.

Protein Purification

The proteins, polypeptides and fragments of this invention can be purified by standard methods, including but not limited to salt or alcohol precipitation, preparative disc-gel electrophoresis, isoelectric focusing, high pressure liquid chromatography (HPLC), reversed-phase HPLC, gel filtration, cation and anion exchange and partition chromatography, and countercurrent distribution. Such purification methods are well known in the art and are disclosed, e.g., in Guide to Protein Purification, Methods in Enzymology, Vol. 182, M. Deutscher, Ed., 1990, Academic Press, New York, N.Y. More specific methods applicable to purification of the SP174 receptors are described below.

Purification steps can be followed by carrying out assays for ligand binding activity as described below. Particularly where a receptor is being isolated from a cellular or tissue source, it is preferable to include one or more inhibitors of proteolytic enzymes is the assay system, such as phenylmethanesulfonyl fluoride (PMSF).

Antibody Production

Antigenic (i.e., immunogenic) fragments of the mammalian SP174 receptors of this invention, which may or may not have ligand binding activity, may similarly be produced. Regardless of whether they bind the SP174 receptor, such fragments, like the complete receptors, are useful as antigens for preparing antibodies by standard methods that can bind to the complete receptors. Shorter fragments can be concatenated or attached to a carrier. Because it is well known in the art that epitopes generally contain at least about five, preferably at least about 8, amino acid residues [Ohno et al., Proc. Natl. Acad. Sci. USA 82:2945 (1985)], fragments used for the production of antibodies will generally be at least that size. Preferably, they will contain even more residues, as described above. Whether a given fragment is immunogenic can readily be determined by routine experimentation.

Although it is generally not necessary when complete mammalian SP174 receptors are used as antigens to elicit antibody production in an immunologically competent host, smaller antigenic fragments are preferably first rendered more immunogenic by cross-linking or concatenation, or by coupling to an immunogenic carrier molecule (i.e., a macromolecule having the property of independently eliciting an immunological response in a host animal). Cross-linking or conjugation to a carrier molecule may be required because small polypeptide fragments sometimes act as haptens (molecules which are capable of specifically binding to an antibody but incapable of eliciting antibody production, i.e., they are not immunogenic). Conjugation of such fragments to an immunogenic carrier molecule renders them more immunogenic through what is commonly known as the “carrier effect”.

Suitable carrier molecules include, e.g., proteins and natural or synthetic polymeric compounds such as polypeptides, polysaccharides, lipopolysaccharides etc. Protein carrier molecules are especially preferred, including but not limited to keyhole limpet hemocyanin and mammalian serum proteins such as human or bovine gammaglobulin, human, bovine or rabbit serum albumin, or methylated or other derivatives of such proteins. Other protein carriers will be apparent to those skilled in the art. Preferably, but not necessarily, the protein carrier will be foreign to the host animal in which antibodies against the fragments are to be elicited.

Covalent coupling to the carrier molecule can be achieved using methods well known in the art, the exact choice of which will be dictated by the nature of the carrier molecule used. When the immunogenic carrier molecule is a protein, the fragments of the invention can be coupled, e.g., using water-soluble carbodiimides such as dicyclohexylcarbodiimide or glutaraldehyde.

Coupling agents such as these can also be used to cross-link the fragments to themselves without the use of a separate carrier molecule. Such cross-linking into aggregates can also increase immunogenicity. Immunogenicity can also be increased by the use of known adjuvants, alone or in combination with coupling or aggregation.

Suitable adjuvants for the vaccination of animals include but are not limited to Adjuvant 65 (containing peanut oil, mannide monooleate and aluminum monostearate); Freund's complete or incomplete adjuvant; mineral gels such as aluminum hydroxide, aluminum phosphate and alum; surfactants such as hexadecylamine, octadecylamine, lysolecithin, dimethyldioctadecylammonium bromide, N,N-dioctadecyl-N′,N′-bis(2-hydroxymethyl) propanediamine, methoxyhexadecylglycerol and pluronic polyols; polyanions such as pyran, dextran sulfate, poly IC, polyacrylic acid and carbopol; peptides such as muramyl dipeptide, dimethylglycine and tuftsin; and oil emulsions. The polypeptides could also be administered following incorporation into liposomes or other microcarriers.

Information concerning adjuvants and various aspects of immunoassays are disclosed, e.g., in the series by P. Tijssen, Practice and Theory of Enzyme Immunoassays, 3rd Edition, 1987, Elsevier, New York. Other useful references covering methods for preparing polyclonal antisera include Microbiology, 1969, Hoeber Medical Division, Harper and Row; Landsteiner, Specificity of Serological Reactions, 1962, Dover Publications, New York, and Williams, et al., Methods in Immunology and Immunochemistry, Vol. 1, 1967, Academic Press, New York.

Serum produced from animals immunized using standard methods can be used directly, or the IgG fraction can be separated from the serum using standard methods such as plasmaphoresis or adsorption chromatography with IgG-specific adsorbents such as immobilized Protein A. Alternatively, monoclonal antibodies can be prepared.

Hybridomas producing monoclonal antibodies against the SP174 receptors of the invention or antigenic fragments thereof are produced by well-known techniques. Usually, the process involves the fusion of an immortalizing cell line with a B-lymphocyte that produces the desired antibody. Alternatively, non-fusion techniques for generating immortal antibody-producing cell lines can be used, e.g., virally-induced transformation [Casali et al., Science 234:476 (1986)]. Immortalizing cell lines are usually transformed mammalian cells, particularly myeloma cells of rodent, bovine, and human origin. Most frequently, rat or mouse myeloma cell lines are employed as a matter of convenience and availability.

Techniques for obtaining antibody-producing lymphocytes from mammals injected with antigens are well known. Generally, peripheral blood lymphocytes (PBLs) are used if cells of human origin are employed, or spleen or lymph node cells are used from non-human mammalian sources. A host animal is injected with repeated dosages of the purified antigen (human cells are sensitized in vitro), and the animal is permitted to generate the desired antibody-producing cells before they are harvested for fusion with the immortalizing cell line. Techniques for fusion are also well known in the art, and in general involve mixing the cells with a fusing agent, such as polyethylene glycol.

Hybridomas are selected by standard procedures, such as HAT (hypoxanthine-aminopterin-thymidine) selection. Those secreting the desired antibody are selected using standard immunoassays, such as Western blotting, ELISA (enzyme-linked immunosorbent assay), RIA (radioimmunoassay), or the like. Antibodies are recovered from the medium using standard protein purification techniques [Tijssen, Practice and Theory of Enzyme Immunoassays (Elsevier, Amsterdam, 1985)].

Many references are available to provide guidance in applying the above techniques [Kohler et al., Hybridoma Techniques (Cold Spring Harbor Laboratory, New York, 1980); Tijssen, Practice and Theory of Enzyme Immunoassays (Elsevier, Amsterdam, 1985); Campbell, Monoclonal Antibody Technology (Elsevier, Amsterdam, 1984); Hurrell, Monoclonal Hybridoma Antibodies: Techniques and Applications (CRC Press, Boca Raton, Fla., 1982)]. Monoclonal antibodies can also be produced using well-known phage library systems. See, e.g., Huse, et al., Science 246:1275 (1989); Ward, et al., Nature 341:544 (1989).

Antibodies thus produced, whether polyclonal or monoclonal, can be used, e.g., in an immobilized form bound to a solid support by well known methods, to purify the receptors by immunoaffinity chromatography.

Antibodies against the antigenic fragments can also be used, unlabeled or labeled by standard methods, as the basis for immunoassays of the mammalian SP174 receptors. The particular label used will depend upon the type of immunoassay. Examples of labels that can be used include but are not limited to radiolabels such as ³²P, ¹²⁵I, ³H and ¹⁴C; fluorescent labels such as fluorescein and its derivatives, rhodamine and its derivatives, dansyl and umbelliferone; chemiluminescers such as luciferia and 2,3-dihydrophthalazinediones; and enzymes such as horseradish peroxidase, alkaline phosphatase, lysozyme and glucose-6-phosphate dehydrogenase.

The antibodies can be tagged with such labels by known methods. For example, coupling agents such as aldehydes, carbodiimides, dimaleimide, imidates, succinimides, bisdiazotized benzadine and the like may be used to tag the antibodies with fluorescent, chemiluminescent or enzyme labels. The general methods involved are well known in the art and are described, e.g., in Immunoassay: A Practical Guide, 1987, Chan (Ed.), Academic Press, Inc., Orlando, Fla. Such immunoassays could be carried out, for example, on fractions obtained during purification of the receptors.

The antibodies of the present invention can also be used to identify particular cDNA clones expressing the SP174 receptors in expression cloning systems.

Neutralizing antibodies specific for the ligand binding site of a receptor can also be used as antagonists (inhibitors) to block the SP174 receptor binding. Such neutralizing antibodies can readily be identified through routine experimentation, e.g., by using the radioligand binding assay described infra. Antagonism of the SP174 receptor activity can be accomplished using complete antibody molecules, or well known antigen binding fragments such as Fab, Fc, F(ab)₂, and Fv fragments.

Definitions of such fragments can be found, e.g., in Klein, Immunology (John Wiley, New York, 1982); Parham, Chapter 14, in Weir, ed. Immunochemistry, 4th Ed. (Blackwell Scientific Publishers, Oxford, 1986). The use and generation of antibody fragments has also been described, e.g.: Fab fragments [Tijssen, Practice and Theory of Enzyme Immunoassays (Elsevier, Amsterdam, 1985)], Fv fragments [Hochman et al., Biochemistry 12:1130 (1973); Sharon et al., Biochemistry 15:1591 (1976); Ehrlich et al., U.S. Pat. No. 4,355,023] and antibody half molecules (Auditore-Hargreaves, U.S. Pat. No. 4,470,925). Methods for making recombinant Fv fragments based on known antibody heavy and light chain variable region sequences have further been described, e.g., by Moore et al. (U.S. Pat. No. 4,642,334) and by Plückthun [Bio/Technology 9:545 (1991)]. Alternatively, they can be chemically synthesized by standard methods.

Anti-idiotypic antibodies, both polyclonal and monoclonal, can also be produced using the antibodies elicited against the receptors as antigens. Such antibodies can be useful as they may mimic the receptors.

Nucleic Acids and Expression Vectors

As used herein, the term “isolated nucleic acid” means a nucleic acid such as an RNA or DNA molecule, or a mixed polymer, which is substantially separated from other components that are normally found in cells or in recombinant DNA expression systems. These components include but are not limited to ribosomes, polymerases, serum components, and flanking genomic sequences. The term thus embraces a nucleic acid which has been removed from its naturally occurring environment, and includes recombinant or cloned DNA isolates and chemically synthesized analogs or analogs biologically synthesized by heterologous systems. A substantially pure molecule includes isolated forms of the molecule.

An isolated nucleic acid will generally be a homogeneous composition of molecules but may, in some embodiments, contain minor heterogeneity. Such heterogeneity is typically found at the ends of nucleic acid coding sequences or in regions not critical to a desired biological function or activity.

A “recombinant nucleic acid” is defined either by its method of production or structure. Some recombinant nucleic acids are thus made by the use of recombinant DNA techniques which involve human intervention, either in manipulation or selection. Others are made by fusing two fragments not naturally contiguous to each other. Engineered vectors are encompassed, as well as nucleic acids comprising sequences derived using any synthetic oligonucleotide process.

For example, a wild-type codon may be replaced with a redundant codon encoding the same amino acid residue or a conservative substitution, while at the same time introducing or removing a nucleic acid sequence recognition site. Similarly, nucleic acid segments encoding desired functions may be fused to generate a single genetic entity encoding a desired combination of functions not found together in nature. Although restriction enzyme recognition sites are often the target of such artificial manipulations, other site-specific targets, e.g., promoters, DNA replication sites, regulation sequences, control sequences, or other useful features may be incorporated by design. Sequences encoding epitope tags for detection or purification as described above may also be incorporated.

A nucleic acid “fragment” is defined herein as a nucleotide sequence comprising at least about 17, generally at least about 25, preferably at least about 35, more preferably at least about 45, and most preferably at least about 55 or more contiguous nucleotides.

This invention further encompasses recombinant DNA molecules and fragments having sequences that are identical or highly homologous to those described herein. The nucleic acids of the invention may be operably linked to DNA segments which control transcription, translation, and DNA replication.

“Homologous nucleic acid sequences” are those which when aligned and compared exhibit significant similarities. Standards for homology in nucleic acids are either measures for homology generally used in the art by sequence comparison or based upon hybridization conditions, which are described in greater detail below.

Substantial nucleotide sequence homology is observed when there is identity in nucleotide residues in two sequences (or in their complementary strands) when optimally aligned to account for nucleotide insertions or deletions, in at least about 50%, preferably in at least about 75%, more preferably in at least about 90%, and most preferably in at least about 95% of the aligned nucleotides.

Substantial homology also exists when one sequence will hybridize under selective hybridization conditions to another. Typically, selective hybridization will occur when there is at least about 55% homology over a stretch of at least about 30 nucleotides, preferably at least about 65% over a stretch of at least about 25 nucleotides, more preferably at least about 75%, and most preferably at least about 90% over about 20 nucleotides. See, e.g., Kanehisa, Nucleic Acids Res. 12:203 (1984).

The lengths of such homology comparisons may encompass longer stretches and in certain embodiments may cover a sequence of at least about 17, preferably at least about 25, more preferably at least about 50, and most preferably at least about 75 nucleotide residues.

Stringency of conditions employed in hybridizations to establish homology are dependent upon factors such as salt concentration, temperature, the presence of organic solvents, and other parameters. Stringent temperature conditions usually include temperatures in excess of about 30° C., often in excess of about 37° C., typically in excess of about 45° C., preferably in excess of about 55° C., more preferably in excess of about 65° C., and most preferably in excess of about 70° C. Stringent salt conditions will ordinarily be less than about 1000 mM, usually less than about 500 mM, more usually less than about 400 mM, preferably less than about 300 mM, more preferably less than about 200 mM, and most preferably less than about 150 mM. For example, salt concentrations of 100, 50 and 20 mM are used. The combination of the foregoing parameters, however, is more important than the measure of any single parameter. See, e.g., Wetmur et al., J. Mol. Biol. 31:349 (1968).

For sequence comparison, typically one sequence acts as a reference sequence, to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optical alignment of sequences for comparison can be conducted, e.g., by the local homology algorithm of Smith and Waterman (1981) Adv. Appl. Math. 2:482, by the homology alignment algorithm of Needlman and Wunsch (1970) J. Mol. Biol. 48:443, by the search for similarity method of Pearson and Lipman (1988) Proc. Nat'l Acad. Sci. USA 85:2444, by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally Ausubel et al., supra).

One example of a useful algorithm is PILEUP. PILEUP creates a multiple sequence alignment from a group of related sequences using progressive, pairwise alignments to show relationship and percent sequence identity. It also plots a tree or dendogram showing the clustering relationships used to create the alignment. PILEUP uses a simplification of the progressive alignment method of Feng and Doolittle (1987) J. Mol. Evol. 35:351-360. The method used is similar to the method described by Higgins and Sharp (1989) CABIOS 5:151-153. The program can align up to 300 sequences, each of a maximum length of 5,000 nucleotides or amino acids. The multiple alignment procedure begins with the pairwise alignment of the two most similar sequences, producing a cluster of two aligned sequences. This cluster is then aligned to the next most related sequence or cluster of aligned sequences. Two clusters of sequences are aligned by a simple extension of the pairwise alignment of two individual sequences. The final alignment is achieved by a series of progressive, pairwise alignments. The program is run by designating specific sequences and their amino acid or nucleotide coordinates for regions of sequence comparison and by designating the program parameters. For example, a reference sequence can be compared to other test sequences to determine the percent sequence identity relationship using the following parameters: default gap weight (3.00), default gap length weight (0.10), and weighted end gaps.

Another example of algorithm that is suitable for determining percent sequence identity and sequence similarity is the BLAST algorithm, which is described in Altschul, et al. (1990) J. Mol. Biol. 215:403-410. Software for performing BLAST analyses is publicly available through the National Center for Biotechnology Information (http:www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul, et al., supra). These initial neighborhood word hits act as seeds for initiating searches to find longer HSPs containing them. The word hits are then extended in both directions along each sequence for as far as the cumulative alignment score can be increased. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLAST program uses as defaults a wordlength (W) of 11, the BLOSUM62 scoring matrix (see Henikoff and Henikoff (1989) Proc. Nat'l Acad. Sci. USA 89:10915) alignments (B) of 50, expectation (E) of 10, M=5, N=4, and a comparison of both strands.

In addition to calculating percent sequence identity, the BLAST algorithm also performs a statistical analysis of the similarity between two sequences (see, e.g., Karlin and Altschul (1993) Proc. Nat'l Acad. Sci. USA 90:5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.1, more preferably less than about 0.01, and most preferably less than about 0.001.

A further indication that two nucleic acid sequences of polypeptides are substantially identical is that the polypeptide encoded by the first nucleic acid is immunologically cross reactive with the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules hybridize to each other under stringent conditions, as described below.

The term “substantially pure” is defined herein to mean a mammalian SP174 receptor, nucleic acid or other material that is free from other contaminating proteins, nucleic acids, and other biologicals derived from an original source organism or recombinant DNA expression system. Purity may be assayed by standard methods and will typically exceed at least about 50%, preferably at least about 75%, more preferably at least about 90%, and most preferably at least about 95% purity. Purity evaluation may be made on a mass or molar basis.

Nucleic acids encoding the SP174 receptors or fragments thereof can be prepared by standard methods. For example, DNA can be chemically synthesized using, e.g., the phosphoramidite solid support method of Matteucci et al. [J. Am. Chem. Soc. 103:3185 (1981)], the method of Yoo et al. [J. Biol. Chem. 764:17078 (1989)], or other well known methods. This can be done by sequentially linking a series of oligonucleotide cassettes comprising pairs of synthetic oligonucleotides, as described below.

Of course, due to the degeneracy of the genetic code, many different nucleotide sequences can encode the SP174 receptors. The codons can be selected for optimal expression in prokaryotic or eukaryotic systems. Such degenerate variants are of course also encompassed by this invention.

Moreover, nucleic acids encoding the SP174 receptors can readily be modified by nucleotide substitutions, nucleotide deletions, nucleotide insertions, and inversions of nucleotide stretches. Such modifications result in novel DNA sequences which encode antigens having immunogenic or antigenic activity in common with the wild-type receptors. These modified sequences can be used to produce wild-type or mutant receptors, or to enhance expression in a recombinant DNA system.

Insertion of the DNAs encoding the SP174 receptors into a vector is easily accomplished when the termini of both the DNAs and the vector comprise compatible restriction sites. If this cannot be done, it may be necessary to modify the termini of the DNAs and/or vector by digesting back single-stranded DNA overhangs generated by restriction endonuclease cleavage to produce blunt ends, or to achieve the same result by filling in the single-stranded termini with an appropriate DNA polymerase.

Alternatively, desired sites may be produced, e.g., by ligating nucleotide sequences (linkers) onto the termini. Such linkers may comprise specific oligonucleotide sequences that define desired restriction sites. Restriction sites can also be generated by the use of the polymerase chain reaction (PCR). See, e.g., Saiki et al., Science 239:487 (1988). The cleaved vector and the DNA fragments may also be modified if required by homopolymeric tailing.

Recombinant expression vectors used in this invention are typically self-replicating DNA or RNA constructs comprising nucleic acids encoding one of the mammalian SP174 receptors, usually operably linked to suitable genetic control elements that are capable of regulating expression of the nucleic acids in compatible host cells. Genetic control elements may include a prokaryotic promoter system or a eukaryotic promoter expression control system, and typically include a transcriptional promoter, an optional operator to control the onset of transcription, transcription enhancers to elevate the level of mRNA expression, a sequence that encodes a suitable ribosome binding site, and sequences that terminate transcription and translation. Expression vectors also may contain an origin of replication that allows the vector to replicate independently of the host cell.

Vectors that could be used in this invention include microbial plasmids, viruses, bacteriophage, integratable DNA fragments, and other vehicles which may facilitate integration of the nucleic acids into the genome of the host. Plasmids are the most commonly used form of vector but all other forms of vectors which serve an equivalent function and which are, or become, known in the art are suitable for use herein. See, e.g., Pouwels et al., Cloning Vectors: A Laboratory Manual, 1985 and Supplements, Elsevier, N.Y., and Rodriguez et al. (eds.), Vectors: A Survey of Molecular Cloning Vectors and Their Uses, 1988, Buttersworth, Boston, Mass.

Expression of nucleic acids encoding the SP174 receptors of this invention can be carried out by conventional methods in either prokaryotic or eukaryotic cells. Although strains of E. coli are employed most frequently in prokaryotic systems, many other bacteria such as various strains of Pseudomonas and Bacillus are know in the art and can be used as well.

Prokaryotic expression control sequences typically used include promoters, including those derived from the -lactamase and lactose promoter systems [Chang et al., Nature 198:1056 (1977)], the tryptophan (trp) promoter system [Goeddel et al., Nucleic Acids Res. 8:4057 (1980)], the lambda P_{L} promoter system [Shimatake et al., Nature 292:128 (1981)] and the tac promoter [De Boer et al., Proc. Natl. Acad. Sci. USA 292:128 (1983)]. Numerous expression vectors containing such control sequences are known in the art and available commercially.

Suitable host cells for expressing nucleic acids encoding the mammalian SP174 receptors include prokaryotes and higher eukaryotes. Prokaryotes include both gram negative and positive organisms, e.g., E. coli and B. subtilis. Higher eukaryotes include established tissue culture cell lines from animal cells, both of non-mammalian origin, e.g., insect cells, and birds, and of mammalian origin, e.g., human, primates, and rodents.

Prokaryotic host-vector systems include a wide variety of vectors for many different species. As used herein, E. coli and its vectors will be used generically to include equivalent vectors used in other prokaryotes. A representative vector for amplifying DNA is pBR322 or many of its derivatives. Vectors that can be used to express the mammalian SP174 receptors include but are not limited to those containing the lac promoter (pUC-series); trp promoter (pBR322-trp); Ipp promoter (the pIN-series); lambda-pP or pR promoters (pOTS); or hybrid promoters such as ptac (pDR540). See Brosius et al., “Expression Vectors Employing Lambda-, trp-, lac-, and Ipp-derived Promoters”, in Rodriguez and Denhardt (eds.) Vectors: A Survey of Molecular Cloning Vectors and Their Uses, 1988, Buttersworth, Boston, pp. 205-236.

Higher eukaryotic tissue culture cells are preferred hosts for the recombinant production of the mammalian SP174 receptors. Although any higher eukaryotic tissue culture cell line might be used, including insect baculovirus expression systems, mammalian cells are preferred. Transformation or transfection and propagation of such cells has become a routine procedure. Examples of useful cell lines include HeLa cells, Chinese hamster ovary (CHO) cell lines, baby rat kidney (BRK) cell lines, insect cell lines, bird cell lines, and monkey (COS) cell lines.

Expression vectors for such cell lines usually include an origin of replication, a promoter, a translation initiation site, RNA splice sites (if genomic DNA is used), a polyadenylation site, and a transcription termination site. These vectors also usually contain a selection gene or amplification gene. Suitable expression vectors may be plasmids, viruses, or retroviruses carrying promoters derived, e.g., from such sources as adenovirus, SV40, parvoviruses, vaccinia virus, or cytomegalovirus. Representative examples of suitable expression vectors include pCR®3.1, pcDNA1, pCD [Okayama et al., Mol. Cell Biol. 5:1136 (1985)], pMC1neo Poly-A [Thomas et al., Cell 51:503 (1987)], pUC19, pREP8, pSVSPORT and derivatives thereof, and baculovirus vectors such as pAC 373 or pAC 610.

Screening Systems and Methods

The SP174 receptor of this invention can be employed in screening systems to identify agonists or antagonists of the receptor. Essentially, these systems provide methods for bringing together a mammalian SP174 receptor, an appropriate known ligand or an analogue thereof and a sample to be tested for the presence of the SP174 receptor agonist or antagonist. As used herein, the term “the SP174 receptor ligand” is defined to mean ADP, ADPβS, 2-MeS-ADP, ATP, 2-MeS-ATP, 2-Cl-ATP, ATPγS, AP3A, IDP or analogues thereof.

Two basic types of screening systems can be used, a labeled-ligand binding assay and a “functional” assay. A labeled ligand for use in the binding assay can be obtained by labeling the SP174 receptor ligand or a known the SP174 receptor agonist with a measurable group as described above in connection with the labeling of antibodies. Various labeled forms of the SP174 receptor ligands are available commercially or can be generated using standard techniques.

Typically, a given amount of the SP174 receptor of the invention is contacted with increasing amounts of a labeled ligand, such as labeled ADP, and the amount of the bound labeled ligand is measured after removing unbound labeled ligand by washing. As the amount of the labeled ligand is increased, a point is eventually reached at which all receptor binding sites are occupied or saturated. Specific receptor binding of the labeled ligand is abolished by a large excess of unlabeled ligand.

Preferably, an assay system is used in which non-specific binding of the labeled ligand to the receptor is minimal. Non-specific binding is typically less than 50%, preferably less than 15%, and more preferably less than 10% of the total binding of the labeled ligand.

In principle, a binding assay of the invention may be carried out using a soluble receptor of the invention, e.g., following production and refolding by standard methods from an E. coli expression system, and the resulting receptor-labeled ligand complex may be precipitated, e.g., using an antibody against the receptor. The precipitate may then be washed and the amount of the bound labeled ligand may be measured.

Preferably, however, a nucleic acid encoding one of the SP174 receptors of the invention is transfected into an appropriate host cell, whereby the receptor becomes incorporated into the membrane of the cell. A membrane fraction can then be isolated from the cell and used as a source of the receptor for assay. Preferably, specific binding of the labeled ligand to a membrane fraction from the untransfected host cell is negligible.

The binding assays of this invention can be used to identify both the SP174 receptor agonists and antagonists, because both compete for binding to the receptor with the labeled ligand.

In the basic binding assay, the method for identifying a SP174 receptor agonist or antagonist comprises:

(a) contacting a mammalian SP174 receptor or a functional fragment thereof in the presence of a known amount of labeled SP174 receptor ligand with a sample to be tested for the presence of the SP174 receptor agonist or antagonist; and

(b) measuring the amount of labeled SP174 receptor ligand bound to the receptor;

whereby the SP174 receptor agonist or antagonist in the sample is identified by measuring substantially reduced binding of the labeled SP174 receptor ligand to the SP174 receptor, compared to what would be measured in the absence of such agonist or antagonist.

Preferably, the SP174 receptor used to identify the SP174 receptor agonist or antagonist for human therapeutic purposes has an amino acid sequence defined by SEQ ID NO: 2 or a subsequence thereof.

Determination of whether a particular molecule inhibiting binding of the labeled ligand to the receptor is an antagonist or an agonist is then determined in a second, functional assay. The functionality of the SP174 receptor agonists and antagonists identified in the binding assay can be determined in cellular and animal models.

In cellular models, parameters for intracellular activities mediated by the SP174 receptor can be monitored for antagonistic and/or agonistic activities. Such parameters include but are not limited to intracellular second messenger pathways activated by the SP174 receptor, changes in cell growth rate, secretion of hormones, etc., using published methods. Examples of the methods are measurement of the effects of the ligands on receptor-mediated inhibition of forskolin-stimulated intracellular cAMP production [Parker et al., Mol. Brain Res. 34:179-189 (1995)], receptor-stimulated Ca⁺⁺ mobilization and mitogenic effects [Sethi et al., Cancer Res. 51:1674-1679(1991)], inositol phosphate production and mitogen activated protein kinase (MAP kinase) induction (Wang et al., Biochemistry 37:6711-17 (1998) and receptor-mediated glucose-stimulated insulin release [Yanaihara et al., Regulatory Peptides 46:93-101 (1993)].

Agonists of the SP174 receptors may also be identified directly by using functional assays. An agonist may or may not directly inhibit the SP174 receptor ligand binding to the SP174 receptor.

Pharmaceutical Compositions

The SP174 receptor agonists and antagonists, such as, for example, small organic molecules, peptides, inhibitory ligand analogs, neutralizing antibodies or binding fragments thereof, as well as other types of agonists and antagonists, which can be identified using the methods of the invention, may be used therapeutically to modulate the activity of the SP174 receptor, and thereby to treat any medical condition caused or mediated by the SP174 receptor.

The dosage regimen involved in therapeutic application will be determined by the attending physician, considering various factors which may modify the action of the SP174 receptor antagonists, e.g., the condition, body weight, sex and diet of the patient, the severity of any infection, time of administration, and other clinical factors.

Although the compositions of this invention may be administered in simple solution, they are more typically used in combination with other materials such as carriers, preferably pharmaceutical carriers. Useful pharmaceutical carriers can be any compatible, non-toxic substance suitable for delivering the compositions of the invention to a patient. Sterile water, alcohol, fats, waxes, and inert solids may be included in a carrier. Pharmaceutically acceptable adjuvants (buffering agents, dispersing agents) may also be incorporated into the pharmaceutical composition. Generally, compositions useful for parenteral administration of such drugs are well known; e.g. Remington's Pharmaceutical Science, 17th Ed. (Mack Publishing Company, Easton, Pa., 1990). Alternatively, compositions of the invention may be introduced into a patient's body by implantable drug delivery systems [Urquhart et al., Ann. Rev. Pharmacol. Toxicol. 24:199 (1984)].

Therapeutic formulations may be administered in many conventional dosage formulation. Formulations typically comprise at least one active ingredient, together with one or more pharmaceutically acceptable carriers. Formulations may include those suitable for oral, rectal, nasal, or parenteral (including subcutaneous, intramuscular, intravenous and intradermal) administration.

The formulations may conveniently be presented in unit dosage form and may be prepared by any methods well known in the art of pharmacy. See, e.g., Gilman et al. (eds.) (1990), The Pharmacological Bases of Therapeutics, 8th Ed., Pergamon Press; and Remington's Pharmaceutical Sciences, supra, Easton, Penn.; Avis et al. (eds.) (1993) Pharmaceutical Dosage Forms: Parenteral Medications Dekker, New York; Lieberman et al. (eds.) (1990) Pharmaceutical Dosage Forms: Tablets Dekker, New York; and Lieberman et al. (eds.) (1990), Pharmaceutical Dosage Forms: Disperse Systems Dekker, New York.

For therapeutic use, the SP174 receptor antibodies and fragments are preferably chimeric or humanized to reduce antigenicity and human anti-mouse antibody (HAMA) reactions. The methodology involved is disclosed, e.g., in U.S. Pat. No. 4,816,397 to Boss et al. and in U.S. Pat. No. 4,816,567 to Cabilly et al. Further refinements on antibody humanization are described in European Patent 451 216 B1.

Typical protocols for the therapeutic administration of antibodies are well known in the art and have been disclosed, e.g., by Elliott et al. [The Lancet 344:1125 (1994)], Isaacs et al. [The Lancet 340:748 (1992)], Anasetti et al. [Transplantation 54:844 (1992)], Anasetti et al. [Blood 84:1320 (1994)], Hale et al. [The Lancet 2:1394 (Dec. 17, 1988)], Queen [Scrip 1881:18 (1993)] and Mathieson et al. [N. Eng. J Med. 323:250 (1990)].

Often, treatment dosages are titrated upward from a low level to optimize safety and efficacy. For example, daily antibody dosages will fall within a range of about 0.01 to 20 mg protein per kilogram of body weight. Typically, the dosage range will be from about 0.1 to 5 mg protein per kilogram of body weight.

Dosages of antigen binding fragments from the antibodies will be adjusted to account for the smaller molecular sizes and possibly decreased half-lives (clearance times) following administration. Various modifications or derivatives of the antibodies or fragments, such as addition of polyethylene glycol chains (PEGylation), may be made to influence their pharmacokinetic and/or pharmacodynamic properties.

### EXAMPLES

The present invention can be illustrated by the following examples. Unless otherwise indicated, percentages given below for solids in solid mixtures, liquids in liquids, and solids in liquids are on a wt/wt, vol/vol and wt/vol basis, respectively. Sterile conditions were generally maintained during cell culture.

Materials and General Methods

Standard methods were used, as described, e.g., in Maniatis et al., Molecular Cloning: A Laboratory Manual, 1982, Cold Spring Harbor Laboratory, Cold Spring Harbor Press; Sambrook et al., Molecular Cloning: A Laboratory Manual, (2d ed.), Vols 1-3, 1989, Cold Spring Harbor Press, NY; Ausubel et al., Biology, Greene Publishing Associates, Brooklyn, N.Y.; or Ausubel, et al. (1987 and Supplements), Current Protocols in Molecular Biology, Greene/Wiley, New York; Innis et al. (eds.) PCR Protocols: A Guide to Methods and Applications, 1990, Academic Press, N.Y.

The polymerase chain reaction (PCR) was carried out using the Clontech protocols. Briefly, PCR was always run with KLENTAQ polymerase, which possesses proof reading activity (Clontech), and a cycling profile of 94° C. for 30 seconds, 55° C. for 30 seconds and 72° C. for 30 seconds (35 cycles). A GC melt reagent (Clontech) at recommended dilution was always used in PCR reactions.

DNA sequencing was performed with ABI Prism dye termination DNA sequencing reagents (Perkin Elmer, Branchburg, N.J.) and an automated sequencing apparatus (Applied Biosystems ABI377 DNA Sequencer). DNA and protein sequence comparisons were performed with DNA* software from DNAstar Inc., Madison, Wis.

All nucleotides were obtained from either Sigma or RBI. Fluorometric Imaging Plate Reader (FLIPR) instrument was from Molecular Device (California). [³H]2-MeS-ADP was custom-synthesized by Amersham Pharmacia. Fluo-3-AM, pluronic acid and 2.5 mM probenecid were from Molecular Probe. Standard medium conditions were used for culturing of HEK293, CHO, and NIH3T3 cells. Scintillation cocktail (Ready Safe™) for aqueous sample was obtained from Beckman Coulter. Chimera G proteins (Ga16, Gq/z, Gq/s, Gq/12, Gq/i, Gq/i3, Gq/o, Gq/truncated) were constructed by replacing the C-terminal five residues of human Gq with the five amino acid residues of the corresponding G protein (Conklin et al., Nature, 363, p274-276). All chimera G proteins were cloned into pCR3.1 vector.

### Example 1

### Identification and Characterization of the SP174 Receptor Ligand

a. Screening Assay

SP174 receptor cDNA was subcloned into the pcDNA3.1 expression vector (Invitrogen) and transiently co-transfected with chimera G protein Gq/i3 into HEK293f cells using lipofectamine (Gibco-BRL). HEK293f cells are the HEK293 cells which are resistant to G-418. Chimera G protein Gq/i3 was constructed by replacing the C-terminus five amino acid residues of human Gqa with the five amino acid residue of human Gi3a (Conklin et al., Nature, 363, p274-276). pcDNA 3.1 vector DNA was also co-transfected with chimera G protein Gq/i3 as a negative control. The transfected cells were seeded into 96 well plates and incubated overnight at 37° C. in a tissue culture incubator. The growth medium was aspirated and replaced with 1001 μl of loading medium (DMEM containing 1% FBS, 1 mM Fluo-3-AM/10% pluronic acid and 2.5 mM Probenecid) and incubated for 1 hour at 37° C. The cells were subsequently washed 3 times with Hank's BSS buffer containing 20 mM HEPES, 2.5 mM Probenecid, and 0.1% BSA using the gentle setting in a Denley cell washer (Denley Instruments). The washed cells were placed in a Fluorometric Imaging Plate Reader (FLIPR) and changes in cellular fluorescence recorded after the addition of various dilutions of tissue extract fractions.

b. Identification of SP174 ligand

The transfected cells were used to screen our in-house ligand collection about 300 compounds by FLIPR assay, and ADP was found to activate specifically SP174 transfected cells. In addition to ADP, other nucleotides such as ADPβs, 2-MeSADP, 2-Cl-ADP, Ap3A and IP etc. were also found to be specifically active on SP174. EC50 values for ADP, ADPβS, 2-MeS-ADP, 2-MeS-ATP, 2-Cl-ATP and IDP were 80, 43, 21, 97, 94, 196, and 812 nM, respectively, and the order of potency was 2-MeS-ATP=2-MeS-ADP>ADP=ADPβS>IDP.

### Example 2

### Agonist/Antagonist Screening Assays

a. FLIPR assay for agonist or antagonists:

The SP174 receptor assay was similar as Example 1 a. The SP174 receptor cDNA was subcloned into the pcDNA3.1 expression vector (Invitrogen) and transiently co-transfected with Gq/i3 into HEK293f cells using lipofectamine (Gibco-BRL). pcDNA 3.1 vector DNA was also co-transfected with Gq/i3 as a negative control. A variety of nucleotides, RBI small moleculars, and in-house peptide collection were screened. Twelve compounds were identified to be the agonists of the SP174 receptor. Their fluorecence change with different amount of the compounds were detected, the data were processed using Prism software to obtain their EC50 value. Other agonists can be identified using similar approaches. To identify antagonists of the SP174 receptor, two-addition approach was adapted. First, the compounds to be screened were added to the SP174 receptor transfected cells, after 5 minutes 100 nM of ADP were added as second addition and the fluorescence changes were recorded. In this assay antagonists inhibit the fluorecence signal of ADP.

b. cAMP assay for agonist or antagonists

The SP174 receptor cDNA was stably transfected into HEK293f cells according standard procedures. The SP174 receptor stable cell line and wild type cell grown on 12-well plates were incubated for 2 hr with 200 μl of medium plus 5 μCi of [3H]adenine/ml. Then 50 μl of Hepes (250 mM, pH 7.5, 50 μM Forskolin, 200 μM IBMX) with the compound to be screened is added to the cells and incubated for 10 minutes at 37° C. Incubations were terminated by addition of 0.8 ml of 5% trichloroacetic acid. [³H]cAMP was purified using Dowex and alumina chromatography as previously described (Harden et al., Mol. Pharmacol. 21:570-580, 1982).

### Example 3

### Agonist/Antagonist Screening by other Assays

Membrane Preparation

Receptor membranes were prepared as follows. HEK293f Cells transfected with a vector containing cDNA encoding the human the SP174 receptor were incubated in DMEM with 10% FBS in 175 mm plates for 3 days in a humidified 5% CO₂ incubator, after which the medium was removed and the cells washed once with PBS (Dulbecco's phosphate buffered saline; Gibco; cat# 14190-144).

Cells are detached from the tissue culture plates by the addition of 5 mls cell dissociation solution (Sigma C5914) per plate. Cells from 10 plates were combined and centrifuged at 1000×g for 5 minutes. The resulting cell pellet was resuspended in 25 ml of 5 mM HEPES (pH 7.4) containing 1×Complete Protease Inhibitor Cocktail (Boehringer Mannheim) and incubated at room temperature for 15 minutes. The cell suspension is then centrifuged for 15 minutes at 4° C. at 11000×g using a Sorvall SS34 rotor. The resulting cell membrane pellet is resuspended in 5 mM HEPES containing 1×Protease Inhibitor Cocktail. The cell suspension is drawn five times through a syringe equipped with a 23 gauge needle, to ensure uniformity of membranes, and frozen in 200 μl aliquots in liquid nitrogen and stored at −80° C.

SP174 Receptor Binding Assay:

Binding of 2-MeS[³H]-ADP (or other radio-labeled ligand) to the membrane preparations was performed in a buffer containing 25 mM HEPES (pH 7.4), 0.1% bovine serum albumin, 10 mM MgCl₂, 10 mM NaCl and 5 mM MnCl₂ (Binding Buffer). Wheat Germ Agglutinin Scintillation Proximity Assay beads (WGA-SPA beads, Amersham, RPNA0001) were resuspended in the Binding Buffer at a concentration of 50 mg/ml. From a stock solution of 1 nM 2-MeS[³H]-ADP in binding buffer 50 μl aliquots are added to each well of a Microlite flat bottomed plate. Then 50 μl of binding buffer (for measurement of total binding), or 50 μl of 4 μM unlabeled 2-MeS[³H]-ADP (for non-specific binding), or 50 μl of sample compound (at 4×the desired final concentration) are added to appropriate wells. 1 mg of membranes (prepared from HEK293f cells stably expressing the SP174 receptor) were preincubated with 10 mg of WGA-SPA beads in 10 ml final volume of binding buffer on ice for 5 minutes. The mixture was centrifuged at 1500×g for 3.5 minutes and the pellet was resuspended in 10 ml fresh binding buffer by gentle mixing. The mixture is again centrifuged at 1500×g for 3.5 minutes and the pellet is again resuspended in 10 ml fresh binding buffer. 100 μl aliquots are added to each well of the 96 well Microlite flat bottomed plate already containing the 2-MeS[³H]-ADP and binding buffer, 2-MeS[³H]-ADP and compound, or 2-MeS[³H]-ADP and unlabelled the SP174 receptor. The plate is incubated at room temperature for 2 hours and the radioactivity measured using a TOPCOUNT beta counter (Packard).

Mitogen Activated Protein (MAP) Kinase Assay:

HEK293f cells stably expressing the SP174 receptors were grown to confluence in 12 well plates and the cells then starved by aspirating the media, adding 1 ml DMEM media (Gibco-BRL) containing 0.1% FBS to each well and incubating for 16-18 hours at 37° C. The medium is then aspirated and replaced by 1 ml F12 media with no sera and incubated at 37° C. for 1 h. The cells were then stimulated for 5 min with ADP (0.1-1000 mM) with sample compounds to be tested for antagonism. When screening for agonists, the cells were stimulated with sample compounds alone. Following stimulation, cells were washed with cold (4° C.) PBS and lysed by the addition of 100 μl of lysis buffer (50 mM Tris-HCl (pH 8), 150 mM NaCl, 1.0% Nonidet P-40, 0.5% sodium deoxycholate, 0.1% SDS, 10 mM NaF) to each well. Cells were then scraped from the plates, transferred to 1.5 ml microfuge tubes and centrifuged for 15 minutes at 15000×g. 15 μl aliquots of the resulting supernatants were subjected to electrophoresis on a 10% Tris-glycine gel (Novex, cat# EC6078). Following electrophoresis, the gel was transferred to nitrocellulose and washed with TTBS (Tween-Tris Buffered Saline: 0.1% Tween; 50 mM Tris pH 7.4; 150 mM NaCl), blocked with 5% nonfat drymilk (1 h), and incubated in primary antibody (New England Biolabs (NEB) MAP kinase kit cat# 9100) to phosphorylated MAP kinase for 16 h (4° C.). The nitrocellulose was then washed with TTBS (3×, 5 min) and incubated with the secondary antibody from NEB MAP kinase kit for 1 h, and washed again with TTBS (3×, 5 min). The detection of secondary antibody by chemiluminensence was performed according to NEB MAP kinase kit instructions.

Inositol Phosphate Assay:

HEK293f cells stably expressing the SP174 receptor were cultured to confluence in 6 or 12 well plates. The cells were then equilibrated for 16-24 hours in 1-2 μCi/ml myo[³H]-inositol (NEN, cat# NET 114A) in complete culture media. The plates were then washed with warm (37° C.) PBS. Cells were stimulated for 45-60 min. at 37 C with agonist (0.01-1000 nM ADP) in the presence or absence of sample compound to be tested for antagonism. When screening for agonists, cells were stimulated with the test compounds alone. The test compounds were dissolved in PBS containing 20 mM LiCl and 1 mM CaCl₂. Reaction was stopped by aspirating the PBS and the cells lysed by adding 1 ml 0.4 M perchloric acid to each well and incubating the plates for 10-15 under refrigeration. 1.0 ml of lysates from wells were added to 0.5 ml of neutralizing solution (0.72 M KOH, 0.6 M KHCO₃) in 12×75 mm tubes. The tubes were shaken and then centrifuged for 5 minutes at 3000×g. Dowex columns of 1 ml of a 50:50 slurry of Dowex in water (Formate form, BioRad AG1-x8 resin, 100-200 mesh cat# AG 140-1444) were prepared and 1 ml samples of the supernatants and 3 ml of water were added to each column. The columns were washed 2×with 10 ml water and then inositol phosphates were eluted from the columns directly into scintillation vials with 4 mls 1.0 M Ammonium formate, 0.1 M formic acid. After the addition of 10 mls scintillation fluid the samples were subjected liquid scintillation spectroscopy (Beckman Scintillation Counter LS3801).

Many modifications and variations of this invention can be made without departing from its spirit and scope, as will be apparent to those skilled in the art. The specific embodiments described herein are offered by way of example only, and the invention is to be limited only by the terms of the appended claims, together with the full scope of equivalents to which such claims are entitled.

What is claimed is:

1. A method for identifying an agonist or antagonist of a mammalian SP174 receptor, comprising:
(a) contacting a mammalian SP174 receptor or a functional fragment thereof, in the presence of a known amount of a labeled SP174 receptor ligand with a sample to be tested for the presence of the SP174 receptor agonist or antagonist; and
(b) measuring the amount of labeled SP174 ligand specifically bound to the receptor;
whereby the SP174 receptor agonist or antagonist in the sample is identified by measuring substantially reduced binding of the labeled SP174 receptor ligand to the receptor, compared to what would be measured in the absence of such agonist or antagonist.

2. The method of claim 1 wherein the SP174 receptor ligand is selected from the group consisting of ADP, ADPβS, 2-MeS-ADP, ATP, 2-MeS-ATP, 2-Cl-ATP, ATPγS, AP3A, IDP or analogues thereof

3. The method of claim 1 wherein the SP174 receptor is characterized by having the amino acid sequence of SEQ ID NO: 2.

4. An agonist or antagonist of a mammalian the SP174 receptor identified by the method of claim 1.

5. A pharmaceutical composition comprising:
(a) an antagonist or agonist of a mammalian the SP174 receptor; and
(b) a pharmaceutically acceptable carrier.

6. The pharmaceutical composition of claim 5 wherein the SP174 receptor agonist or antagonist is a small organic molecule which specifically binds to a mammalian SP174 receptor.

7. The pharmaceutical composition of claim 5 wherein the SP174 receptor antagonist is an antibody or antigen-binding fragment thereof which specifically binds to a mammalian the SP174 receptor and blocks the binding of the SP174 thereto.

8. A method of treating a medical condition caused or mediated by a mammalian SP174 receptor comprising administering to a mammal afflicted with the condition, an effective amount of a pharmaceutical composition comprising an antagonist or agonist of the SP174 receptor.

9. The method of claim 8 wherein the medical condition is a neurodegenerative disorder.

10. The method of claim 9 wherein the neurodegenerative disorder is selected from the group consisting of Parkinson's Disease, Alzheimer's Disease, Huntington's Disease, amyotrophic lateral sclerosis (ALS) and multiple sclerosis (MS).

11. The method of claim 8 wherein the mammal is a human being.

12. The method of claim 8 wherein the medical condition is a cardiovascular disease or a condition related to platelet aggregation.


SEQUENCE LISTING (2 sequences)

SEQ ID NO: 1 - DNA, 1002 bp, Homo Sapiens
atgaacaccacagtratgcaaggcttcaacagatctgagcggtgccccagagacactcggatagtacagctggtattcccagccctctacacagtggttttcttgaccggcatcctgctgaatactttggctctgtgggtgtttgttcacatccccagctcctccaccttcatcatctacctcaaaaacactttggtggccgacttgataatgacactcatgcttcctttcaaaatcctctctgactcacacctggcaccctggcagctcagagcttttgtgtgtcgtttttcttcggtgatattttatgagaccatgtatgtgggcatcgtgctgttagggctcatagcctttgacagattcctcaagatcatcagacctttgagaaatatttttctaaaaaaacctgtttttgcaaaaacggtctcaatcttcatctggttctttttgttcttcatctccctgccaaatatgatcttgagcaacaaggaagcaacaccatcgtctgtgaaaaagtgtgcttccttaaaggggcctctggggctgaaatggcatcaaatggtaaataacatatgccagtttattttctggactgtttttatcctaatgcttgtgttttatgtggttattgcaaaaaaagtatatgattcttatagaaagtccaaaagtaaggacagaaaaaacaacaaaaagctggaaggcaaagtatttgttgtcgtggctgtcttctttgtgtgttttgctccatttcattttgccagagttccatatactcacagtcaaaccaacaataagactgactgtagactgcaaaatcaactgtttattgctaaagaaacaactctctttttggcagcaactaacatttgtatggatcccttaatatacatattcttatgtaaaaaattcacagaaaagctaccatgtatgcaagggagaaagaccacagcatcaagccaagaaaatcatagcagtcagacagacaacataaccttaggctga

SEQ ID NO: 2 - protein, 333 aa, Homo Sapiens
MNTTVMQGFNRSERCPRDTRIVQLVFPALYTVVFLTGILLNTLALWVFVHIPSSSTFIIYLKNTLVADLIMTLMLPFKILSDSHLAPWQLRAFVCRFSSVIFYETMYVGIVLLGLIAFDRFLKIIRPLRNIFLKKPVFAKTVSIFIWFFLFFISLPNMILSNKEATPSSVKKCASLKGPLGLKWHQMVNNICQFIFWTVFILMLVFYVVIAKKVYDSYRKSKSKDRKNNKKLEGKVFVVVAVFFVCFAPFHFARVPYTHSQTNNKTDCRLQNQLFIAKETTLFLAATNICMDPLIYIFLCKKFTEKLPCMQGRKTTASSQENHSSQTDNITLG